(19) United States
(12) Patent Application Publication
(10) Pub. No.: US 2003/0220655 A1
(43) Pub. Date: Nov. 27, 2003

(54) Obstetric forceps and methods for use thereof

(76) Inventor: Carl Hans Rose, Jackson, MS (US)

Correspondence Address: Carl Hans Rose, 1237 Riverside Drive, Jackson, MS, 39202, US

(21) Appl. No.: 10/444,565

(22) Filed: May 23, 2003

Related U.S. Application Data
(60) Provisional application No. 60/383,154, filed on May 24, 2002.

(51) Int. Cl. A61B 17/42

(52) U.S. Cl. 606/122; 606/191

(57) ABSTRACT

Provided herein are calibrated obstetrical forceps and methods for using the same. The forceps include two elongate members, each having a fetal engaging end, a handle end, and a shank connecting the two. On at least one of the shanks are calibration markings that may be used to determine the amount of force being applied to the fetal skull. Such markings can be used by birth attendants to gauge the success or failure of an assisted delivery.

### TECHNICAL FIELD OF THE INVENTION

This invention relates to an obstetric forceps, and more particularly, to an improved obstetric forceps having a means to measure and thereby control the amount of traction applied to the fetus during use. This invention also relates to methods of using the improved calibrated obstetric forceps of the present invention.

### BACKGROUND OF THE INVENTION

Obstetrical forceps have been used to assist women during childbirth for the past several hundred years. Invented in the 16^{th} century, they remained a secretive instrument until coming into widespread use in the early 1800's.

Many different types of forceps have been devised. Most are constructed of steel and when properly applied, will firmly engage the fetus, most commonly the fetal skull, without slippage.

Generally, obstetric forceps consist of a pair of interlocking tools designed to mechanically assist in the delivery of a fetus during labor. When used in their conventional form, each tool is usually composed of a blade portion (either fenestrated or non-fenestrated), a shank, an articulating lock, and a handle portion.

Obstetrical forceps most commonly work by engaging the fetal head while the fetus is still inside the body of the mother. The forceps are then used to either turn the fetal head to a new position wherein the baby can be born more easily and/or to exert tractional or rotational force on the fetal head to pull the baby down through the birth canal to effect delivery.

The amount of traction or pressure applied to the fetal skull is of great concern and is extremely operator-dependent, with no current manner to measure the exact force being applied to the fetal skull. Potential injuries to the baby from the forces of the forceps pulling on the fetal head include brain, scalp, or facial injuries. Abandonment of an attempted delivery is likewise operator-dependent, with no accepted parameters for termination.

Residency programs teach young physicians how to control the amount of force when utilizing obstetric forceps through experience and clinical judgement. Because of the inherent nature of human judgment, miscalculations inevitably occur, leading to potentially life-long injuries for infants. No standards exist for appropriate amount of traction that should be applied during delivery. Thus, the only safety factor is the skill of the birth attendant operating the forceps.

Attempts to prevent the birth attendant from applying too much pressure to the fetal head have been largely unsuccessful. Such attempts include mounting a pressure sensor on the blade portion of the forceps to indicate the pressure being applied and having a shiftable handle which will disengage after a pre-set force has been exceeded. See U.S. Pat. Nos. 3,785,381 and 3,789,949. In the first case, the forceps does not prevent applying the excessive force; it merely indicates when one is applied. The skill of the birth attendant is still a principal factor in the safe use of the forceps. In the latter case, the mechanism restricting the compressive force is complicated, making it relatively expensive to manufacture and maintain. While the device described in U.S. Pat. No. 3,665,925 is of similar intent, its complexity and requirement for assembly prior to use makes it cumbersome for clinical applications and unwieldy in emergent situations.

Accordingly, there is a clear and present need for an obstetrical forceps and method for using the same wherein the amount of pressure applied to the fetus can be accurately determined and modified accordingly. Furthermore, such forceps should be simple in design and construction for practical clinical use.

### SUMMARY OF THE INVENTION

The present invention provides a calibrated obstetrical forceps which allows for precise measurement of the amount of traction applied to the fetal skull during a delivery attempt. The present invention also provides methods for using such improved forceps.

One object of the present invention is to provide birth attendants with better information regarding the amount of force being applied to the fetal skull so that the attendant can decide whether to continue with a particular delivery or abandon the delivery attempt for fear of fetal injury.

Another object of the present invention is to enable users to establish acceptable and unacceptable standards for traction applied to the fetal skull.

Various other objects, advantages, details, and modifications of the present invention will become apparent in the description of the figures and the preferred embodiments of the invention discussed below.

### BRIEF DESCRIPTION OF THE DRAWINGS

FIG. 1 is a depiction of an embodiment of the calibrated forceps of the present invention.

FIG. 2 is a depiction of an embodiment of the calibrated forceps of the present invention.

FIG. 3 is a depiction of an embodiment of the calibrated forceps of the present invention.

### DETAILED DESCRIPTION OF THE INVENTION

Generally speaking, the calibrated forceps of the present invention consists of a set of conventional blades (either fenestrated or non-fenestrated), shanks, a sliding lock and a moveable handle. The handle is constructed to slide on the shank and is anchored internally by a spring of known tension constant between the end of the handle and the shank. Preferably, each side of the forcep pair may be constructed in a similar manner so the compete handle (comprising of right and left sections) would slide as a unit. Preferably, calibration markings are inscribed along the shank so as the forceps are articulated and traction is applied, the handle slides along the shank to expose the marking designating amount of force being applied

In practice, the forceps of the present invention may be used in a similar manner to conventional forceps. In one embodiment, the handle will slide along the shank relative to the amount of traction applied and expose the calibration markings so the operator can determine exactly how much force is being applied to the fetal skull. The forceps operator or birth attendant can then determine if the delivery should be continued or abandoned.

The calibrated forceps of the present invention may be constructed of any material having sufficient durability, sterilizibility, and strength to perform the necessary functions of the forceps, principally to assist in the delivery of a baby from its mother. Suitable materials of construction include steel, all grades of stainless steel, various other metals and alloys, plastics, ceramics, composites, as well as other materials known to be suitable to those skilled in the art. Preferably, the calibrated forceps of the present invention are constructed of steel or a derivative of a polycarbonate plastic. Also, preferably, the calibrated forceps of the present invention are constructed so that it may be disassembled easily for cleaning, repair, or other similar procedures.

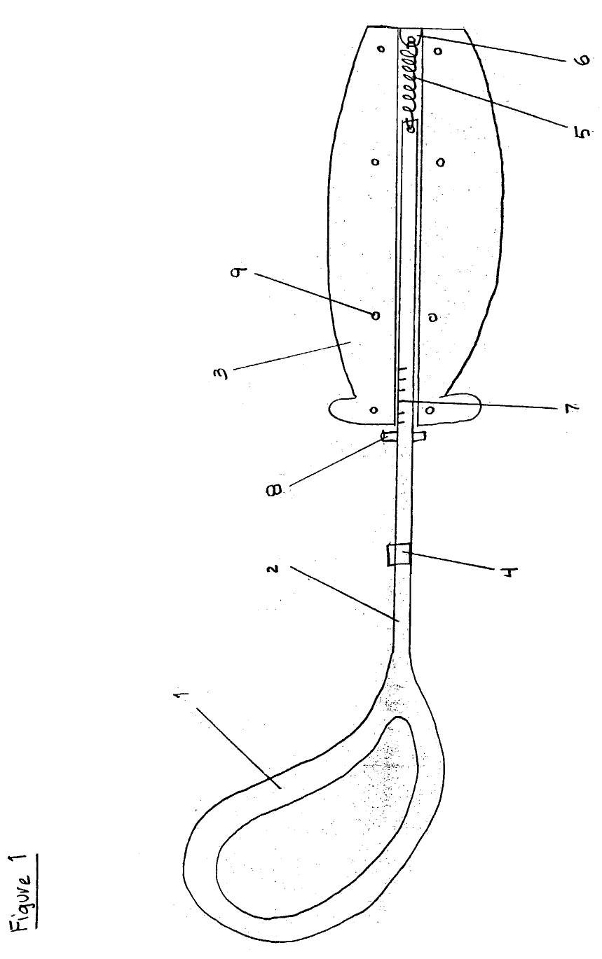

FIG. 1 illustrates one embodiment of the calibrated obstetrical forceps of the present invention. Each part of the set is composed of a blade 1, a shank 2, and a handle 3. An articulating lock 4 would be affixed to the left member of the set. Separate handle 3 is anchored by a spring 5 between the shank and internal mounting tab 6 and slides along the shank with traction to expose calibration markings 7. Stop pin 8 prevents forward travel of the handle at preset point. Alignment pins 9 inset into the right handle interlock with adjoining holes in the left handle to integrate the handle sides into a single contiguous unit.

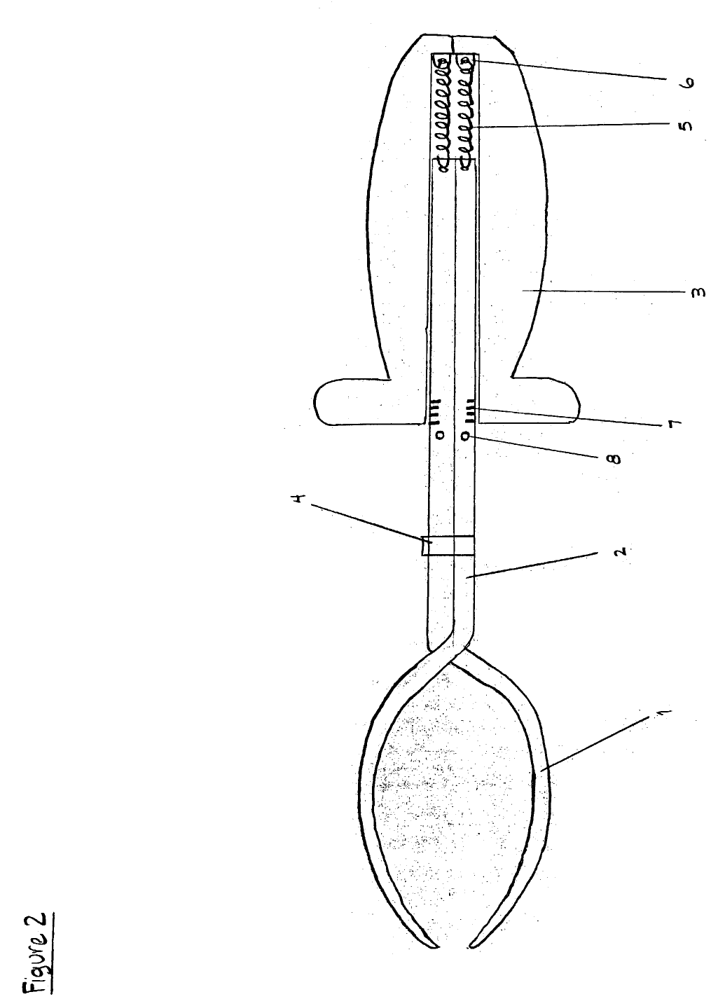

FIG. 2 is an alternative embodiment of the calibrated forceps of the present invention. In this cutaway view, both handles 3 align longitudinally so the shanks 2 are parallel with one another and articulate at sliding lock 4. When traction is applied, the handles then move as a single assembly along the shanks at a rate proportional to force constant of the spring 5.

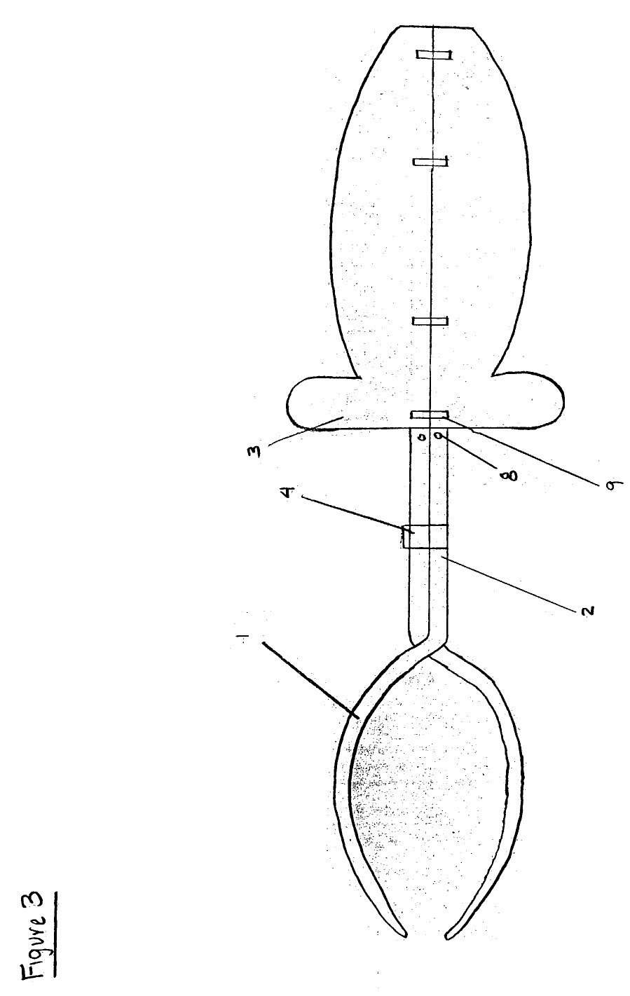

FIG. 3 is an alternative embodiment of the calibrated forceps of the current invention. In this top view, the interlocking pins 9 align each handle with its opposite member to allow the handle 3 to slide along the shanks 2 as a single unit.

In a preferred embodiment, the calibrated forceps of the present invention can be used in the following manner. After decision is made to attempt operative vaginal delivery and fetal head position has been ascertained, the left member is introduced into the birth canal in a conventional manner, coming to rest alongside the fetal skull. The right member is then introduced in a similar manner, and the two shanks are articulated at the lock. Once proper application has been confirmed, traction is applied along the handle assembly in synchrony with maternal expulsive effort. With increasing traction, the handle assembly begins to slide along the shanks exposing the calibration markings, thus denoting the amount of force being applied. Decision can then be made depending on amount of force required if delivery is feasible or should be abandoned for fear of undue injury to fetus or mother.

Another embodiment of the current invention includes a means for determining rotational force applied to the fetal skull during forceps use.

While we have shown and described a present preferred embodiment of the calibrated forceps of the present invention and methods for using same, it is to be distinctly understood that the invention is not limited thereto, but may be otherwise variously embodied within the scope of the following claims.

I claim:

1. Obstetric forceps comprising:
a pair of elongated members, each of which has a fetal engaging blade portion at one end thereof, a handle portion at the opposite end thereof, and a shank connecting said blade portion to said handle portion, said members being connected to each other at a lock and freely moveable with respect to each other when so connected, and wherein calibration markings are inscribed along the shank of at least one of the members.

2. A method of using obstetrical forceps comprising:
(a) providing obstetrical forceps, the obstetrical forceps comprising:
a pair of elongated members, each of which has a fetal engaging blade portion at one end thereof, a handle portion at the opposite end thereof, and a shank connecting said blade portion to said handle portion, said members being connected to each other at a lock and freely moveable with respect to each other when so connected, and wherein calibration markings are inscribed along the shank of at least one of the members
(b) inserting the obstetrical forceps into a vagina of a mother to grasp a fetal head of a baby;
(c) engaging the fetal head with the blade portions of the elongated members; and
(d) examining the calibration markings on the shank of one of the members to determine the amount of force applied to the fetal head.
